(19) United States
(12) Patent Application Publication
(10) Pub. No.: US 2008/0206141 A1
(43) Pub. Date: Aug. 28, 2008

(54) Optical Imaging Contrast Agents

(76) Inventor: Edvin Wilhelm Johannesen, Oslo (NO)

Correspondence Address: GE HEALTHCARE, INC., IP DEPARTMENT, 101 CARNEGIE CENTER, PRINCETON, NJ, 08540-6231, US

(21) Appl. No.: 11/995,220

(22) PCT Filed: Jul. 10, 2006
(86) PCT No.: PCT/NO2006/000264

§ 371 Date: Jan. 10, 2008

(30) Foreign Application Priority Data
Jul. 11, 2005 (NO) 20053354

(51) Int. Cl. A61K 49/00; C07K 14/00; C07K 9/00

(52) U.S. Cl. 424/9.1; 530/326; 530/322

(57) ABSTRACT

The invention relates to optical imaging contrast agents. More specifically the invention relates to optical imaging activatable contrast agent for use in diagnosis and for monitoring the effect of treatment. The contrast agent employs a combined targeting and activation approach and comprises a target binding ligand (V), an enzyme cleavable group (E), a fluorophore (D) and a quencher agent (Q) covalently linked in one molecule.

This invention relates to optical imaging contrast agents. More specifically the invention relates to activatable optical imaging contrast agent for use in diagnosis and for monitoring the effect of treatment.

Optically based imaging methods, and contrast agents used in such methods, have advanced over the last decades. Several methods and techniques based on interaction with light in the electromagnetic spectrum from ultraviolet to near-infrared exist. A range of types of optical imaging contrast agents have also been described, having different properties and for various uses.

Methods using optical imaging contrast agents comprising ligands having affinity for a biological target have been described. For instance, WO 03/011106 discloses compounds comprising antibodies conjugated with photoactive molecules to target biological receptors. Further, U.S. Pat. No. 6,217,848 discloses cyanine and indocyanine chromophore conjugates, including cyanine chromophores linked to bioactive peptides, proteins, oligosaccharides etc. In such a conventional receptor targeting approach there is a risk that the signal from the target compared to background signal is non-optimal due to signal from non-specifically bound or circulating contrast agents.

Other documents describe optical imaging contrast agents that are activated in vivo when interacting with a biological target. WO 02/056670 describes activatable imaging probes that include a plurality of chromophores linked to a chromophore attachment moiety, wherein the optical properties of the chromophores are altered upon activation of the imaging probe. In one embodiment, enzymes can activate the probe by cleavage of this moiety. A potential problem for enzyme activatable contrast agents however, is that the activated contrast agent is being washed away from the biological region of interest after activation, providing less than optimal specificity and hence a low target/background signal ratio.

WO 05/030254 discloses conjugates for detection and diagnosis including a fluorophore linked to a targeting moiety and a quenching agent in such a way that activation of the fluorophore is quenched unless the targeting moiety is bound to a target.

For some indications there is a challenge connected to the fact that the concentration of a biological target molecule can be relatively low. And for oncology there is a challenge to image still smaller tumours. The combination of these challenges makes it attractive to construct contrast agents that are even more specific and which provide an improved target/background signal ratio. There is hence a clinical need for development of improved optical imaging contrast agents which provide an increased target/background signal ratio and increased specificity and sensitivity.

In view of these needs the present invention provides improved contrast agents for optical imaging. The contrast agents of the present invention are designed to provide an improved target/background signal ratio and to provide increased specificity and sensitivity.

Viewed from one aspect the invention provides a dual targeting optical imaging contrast agent comprising;
a target binding ligand (V),
an enzyme cleavable group (E),
a fluorophore (D) and
a quencher agent (Q)
   conjugated with each other in one molecule.

The contrast agents of the present invention employ a combined targeting and activation approach by comprising both a conventional target binding ligand and an enzyme cleavable group. The contrast agents are designed for diseases where one receptor and one enzyme are co-jointly over-expressed in the same tissue or cells. The contrast agent of the present invention reacts with two types of biological targets, a receptor and an enzyme, and this increases the specificity and sensitivity of the contrast agent, compared to contrast agents of the state of the art, reacting with only one type of biological targets. The contrast agent comprises a target binding ligand, an enzyme cleavable group, a fluorophore and a quencher agent linked in such a way that the fluorophore is quenched unless and until the contrast agent is activated. The contrast agent in the non-activated form, as administered, is hence non-fluorescent due to interactions between the fluorophore and the quencher agent. The contrast agent is designed to be activated in vivo by an over-expressed biological enzyme through a reaction with the enzyme cleavable group of the contrast agent. This activation includes a cleaving of the contrast agent into two parts, separating the fluorophore and the quencher agent and de-quenching takes place. The target binding ligand, which is fluorescent after the activation, will concurrently bind to an over-expressed receptor associated with a given disease. As a result of using the dual targeting contrast agent the signal from the background will be low and washout e.g. from the extracellular matrix is prevented. The non-activated contrast agent will be quenched (dark) while the activated contrast agent will stay put in the biological region of interest.

In a second embodiment of the invention the contrast agent comprises the building blocks

### i) E-Q and

### ii) V-D

conjugated with each other, wherein
E represents an enzyme cleavable group,
Q represents a quencher agent,
V represents a target binding ligand and
D represents a fluorophore.

The contrast agent further comprises optional linker moieties connecting the moieties of the building blocks together and connecting the two building blocks together. The E-Q building block is preferably linked to the V-D block via E, optionally via a linker.

The enzyme cleavable group, E, comprises an activation site that will react with a given enzyme resulting in enzymatic cleavage of the contrast agent. The reaction will cause the quencher agent and the fluorophore to be separated. Reaction of the enzyme cleavable group with an enzyme under conditions suitable to cause cleavage of the Enzyme cleavable group-Quencher agent (E-Q) building block from the Target binding ligand-Fluorophore (V-D) building block, modulates the fluorescence properties of the fluorophore, and thereby switches the fluorophore from a first fluorescent state to a second fluorescent state. The contrast agent hence works as a reporter for detecting biological cleavage events and as an identifier for a certain enzyme. When the contrast agent has been cleaved by an enzyme the Target binding ligand-fluorophore (V-D) building block is free to bind to receptors towards which the target binding ligand has affinity. The contrast agent hence also works as a reporter for detection of certain biological receptors.

The contrast agent is preferably constructed in such a way that the Target binding ligand-Fluorophore (V-D) block is prevented from binding to the receptor in the non-activated state, i.e. before the enzyme has cleaved the contrast agent, This will ensure that the enzyme is allowed to perform its action. This prevention of binding in non-activated form is achieved by including e.g. some form of steric hindrance or appropriate linker or bridges between the two building blocks. The contrast agent can be constrained and thereby form a steric hindrance for example by formation of one or more cyclising bridges. A monocyclic peptide compound can be obtained by formation of a disulphide bond or a thioether bond between amino acids. The term “cyclising bridges” refers to any combination of amino acids with functional groups which allows for the introduction of a bridge. Some preferred examples are disulphides, disulphide mimetics such as the —(CH₂)₄— carba bridge, thioacetal, thioether bridges (cystathione or lanthionine) and bridges containing esters and ethers.

In a third embodiment the invention provides a contrast agent of formula (I):

Q-L¹-E-L²-V-L³-D (I)

wherein Q, D, E and V are as hereinbefore defined and
L¹, L² and L³ are all linker moieties which are the same or different.

In a fourth embodiment the invention provides a contrast agent of formula (II):

Q-L¹-E-L²-D-L³-V (II)

wherein Q, D, E, V, and L¹, L² and L³ are as hereinbefore defined.

The contrast agent includes an enzyme cleavable group, E. Suitably, group E comprises a substrate for a hydrolytic enzyme. The enzymes for which the enzyme-cleavable groups are substrates should be over-expressed in specific disease states. The enzyme activity must remain associated with the diseased tissue. Frequently, the enzymes will remain bound to the surface of cells by virtue of being transmembrane proteins or possessing membrane anchors, but the enzymatic activity may also remain localised as a result of the enzyme being inhibited outside the diseased tissue. For instance, matrix metalloproteinases are inhibited by tissue inhibitors of metalloproteinases, and thrombin and plasmin are also inactivated by specific inhibitors in locations where they are not needed.

Suitably, group E comprises a substrate for an enzyme selected from the group of proteases, peptidases, esterases, phosphatases, phosphodiesterases, dealkylases and glycosidases or endoglycanases. Most preferably, E comprises a substrate for a protease or peptidase.

In one embodiment, E comprises a phosphate ester linkage having one or more phosphate groups of the structure:

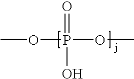

wherein j is an integer from 1 to 4. In this embodiment, E is capable of being cleaved by a phosphatase such as a alkaline phosphatase, or acid phosphatase. The phosphate ester may be a pre-synthesised substrate or may be generated in situ by chemical hydrolysis or by an enzyme catalysed nucleoside monophosphate or nucleoside polyphosphate transfer from a terminal-phosphate labelled nucleoside polyphosphate having the structure:

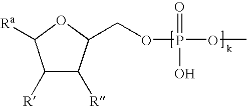

wherein R′ and R″ are independently selected from H and OH; R^{a} is a nucleoside base selected from adenine, guanine, cytosine, thymine, uracil, hypoxanthene and xanthene; and k is an integer from 1 to 6.

In another preferred embodiment, E comprises at least one peptide linkage (—CO—NH—) covalently bonded to Q and D or V, optionally via linkers. In this embodiment, E typically has the structure:

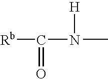

wherein R^{b} is a residue of a peptide or protein. Upon hydrolysis by a peptidase or protease, E is cleaved separating the fluorophore from the quencher agent, and energy is transferred between the fluorophore and quencher agent, allowing detection of an increase in fluorescence emission from the fluorophore.

In a further embodiment, E comprises a glycosidic linkage and is a substrate for a glycosidase such as α-glycosidases (e.g., α-amylase), β-glycosidases (e.g. β-glucosidase) comprising one or more moieties of the structure:

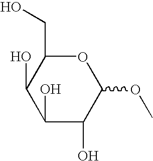

wherein any of the hydroxyl groups is an optional linking site.

In a further embodiment, E comprises an ether linkage that is a substrate for a dealkylase and having the structure:

R^{c}—O—

wherein R^{c} is a C₁-C₂₀ straight or branched chain alkyl.

In a preferred embodiment E is a substrate for any of the enzymes selected from the groups:
A matrix metalloproteinase (MMP), e.g. MMP-2, MMP-3, MMP-7, MMP-9, MMP-14;
A Cathepsin, e.g. Cathepsin B, D, K or L;
A Kallikrein;
A Proproteine convertase;
A Membrane bound serine protease.

In a preferred embodiment the enzyme cleavable group, E, comprises a peptide sequence, and this may comprise both natural, unnatural or modified amino acids. In this preferred embodiment the enzyme cleavable group of the contrast agent comprises either of the following amino acid sequences:
A short peptide, such as of 1-5 amino acids, that comprises a lysine or arginine residue. These amino acid sequences are cleaved at the C-terminal end of the basic amino acid by cathepsin B and K, and by hepsin and some hepsin-related serine proteases;
Arg-Gly-Phe-Phe-Leu- or Arg-Gly-Phe-Phe-Pro-, which are cleaved by cathepsin D;
Pro-Phe-Arg-B or -Val-Leu-Arg-B, which is cleaved by kallikreins,
Ala-Ala-Phe- or Ala-Gly-Leu-Ala-, which are cleaved by a neutral endopeptidase;
Pro-Lys-Pro-Gln-Gln-Phe-Phe-Gly-Leu-Lys-Gly, which is cleaved by MMP-2;
Arg-Pro-Lys-Pro-Val-Glu-Nva-Trp-Arg-Lys-NH₂, which is cleaved by MMP-3;
ProOH-Gly-Pro-Gln-Gly-Phe-Gln-Gly-AsN-ProOH-Gly, which is cleaved by MMP-9;
Leu-Arg-Leu-Ser-Ser-Tyr-Tyr-Ser-Gly, which is cleaved by prostate-specific antigen, a member of the kallikrein family of serine proteases;
Arg-Leu-Ser-Ile-B, which is cleaved by prostase, a member of the kallikrein family of serine proteases;
Phe-Arg-Arg-B, which is cleaved by cathepsin L;
Gly-Pro-Arg-B, which is cleaved by human kallikrein 5 a member of the kallikrein family of serine proteases;
   wherein B is any amino acid or other suitable group selected from Q, D, V, and L¹ and L² and wherein amino acids written in italics represent D-amino acids;.

The dual targeting contrast agent comprises one quenching agent and one fluorophore, Q and D respectively, which are both preferably chromophore moieties. As used herein, a “chromophore” refers to those groups that have favourable absorption characteristics, i.e. are capable of excitation upon irradiation by any of a variety of photonic sources. Chromophores can be fluorescing or non-fluorescing. A “fluorophore” refers to a fluorescent compound, such as a fluorescing chromophore. Suitably, Q and D are linked such that, under suitable conditions, fluorescence resonance energy transfer (FRET) may take place. FRET is a distance-related process in which the electronic excited states of two chromophore molecules interact without emission of a photon. See, Forster, T., “Intermolecular Energy Transfer and Fluorescence”, Ann. Physik., Vol. 2, p. 55, (1948). One result of this interaction is that excitation of a donor molecule enhances the fluorescence emission of an acceptor molecule. The fluorescence quantum yield of the donor is correspondingly diminished. For FRET to occur, suitably, the donor and acceptor chromophore molecules must be in close proximity (typically between 10-100 Å), since energy transfer efficiency decreases inversely as the 6^{th} power of the distance between the donor and acceptor molecules. Suitably, in the present invention, Q is an acceptor chromophore and D is a donor chromophore in the FRET relationship. By donor, it is meant that the chromophore moiety is capable of absorbing energy from light and emitting light at wavelength which are at least partly within the absorption spectrum of the acceptor. By acceptor, it is meant that the chromophore molecule is capable of absorbing energy at a wavelength emitted by a donor chromophore molecule. Suitably, there is overlap between at least a portion of the emission spectrum of the donor chromophore molecule with the absorption spectrum of the acceptor chromophore molecule.

In a preferred embodiment, the quencher agent Q, the acceptor, does not demonstrate significant emission, and more preferably Q is a non-fluorescent chromophore. Upon excitation of the non-fluorescent chromophore, energy is dissipated as heat rather than fluorescence energy and resonance energy transfer or fluorescence emission cannot take place. In this embodiment the contrast agent comprises a fluorophore and a non-fluorescent acceptor chromophore, the latter acting as a quencher agent, which constitute an energy transfer relationship. The fluorescence emission of the donor is reduced through quenching by the acceptor. The use of non-fluorescent chromophores as quencher agents minimizes the intensity of emission from the matched donor chromophore, prior to cleavage of the agent. When resonance energy transfer is lost through separation of the fluorophore and the quencher agent, the fluorescence of the fluorophore is restored.

In principle, any fluorophore may be used for forming the contrast agent of the present invention, provided that the fluorophore contains, or has attached to it, at least one reactive or functional group capable of forming a linkage to the target binding ligand, and in one embodiment also to E, of the contrast agent, optionally via a linker moiety. Suitably, the fluorophore is selected from the group of coumarin dyes, benzocoumarin dyes, xanthene dyes, phenoxazine dyes, rhodamines dyes, acridone dyes, merocyanine dyes, cyanine dyes and derivatives of the bis-pyrromethine boron difluoride chromophores, wherein the fluorophore is capable of transferring energy to the acceptor dye. Suitable xanthene dyes include but are not limited to fluorescein and its derivatives, such as 5-carboxyfluorescein, 6-carboxyfluorescein and 6-carboxy-4′,5′-dichloro-2′,7′-dimethoxyfluorescein. A further group of usable fluorochromes are amino acids having delocalised electrons in aromatic systems, such as fenylalanins, tyrosins and tryptophans. In one embodiment, the fluorophore may be conjugated to a metal surface such as a solid metal nanoparticle or a metal coated nanoparticle whereby fluorescence is enhanced by the phenomenon called surface plasmon field-enhanced fluorescence as described by CD Geddes and JR Lakowicz, J. Fluorescence 12, 131-139, 2002. Examples of such nanoparticles are gold and silver nanoparticles. The quencher agent for a surface-enhanced fluorescent metal nanoparticle may be a classical quencher molecule (non-emitting absorber).

Preferably, the fluorophore is a xanthene dye or a cyanine dye. Even more preferred are the cyanine dyes selected from the groups of carbacyanines, oxacyanines, thiacyanines and azacyanines.

Particularly preferred fluorophores for use in the present invention are cyanine dyes having the general formula (III):

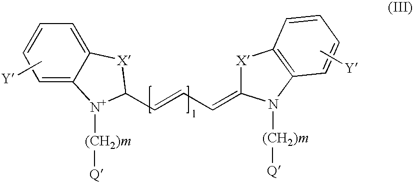

wherein either of X′, Y′ and Q′ includes a reactive or functional group G suitable for attaching to the target binding ligand V. Such group reacts with a complementary group of the target bonding ligand, with the formation of a covalent linkage between the fluorophore D and the target bonding ligand. X′, Y′ or Q′ may hence include a reactive group that may react with a complementary functional group of the target bonding ligand, or alternatively may include a functional group that may react with a reactive group of target bonding ligand. Examples of reactive and functional groups include succinimidyl ester, sulpho-succinimidyl ester, 4-sulfo-2,3,5,6-tetrafluorophenol (STP) ester, isothiocyanate, maleimide, haloacetamide, acid halide, hydrazide, vinylsulphone, dichlorotriazine, phosphoramidite, hydroxyl, amino, sulphydryl, carbonyl, carboxcylic acid and thiophosphate. Preferably G is an ester and more preferably succinimidyl ester.

In one embodiment,

X′ is independently selected from the group of —C(CH₃)₂, sulphur, oxygen,
C(CH₂)ₐCH₃(CH₂)_{b}M, wherein a is an integer of from 0 to 5, b is an integer of 1 to 5, and M is group G or is selected from the group of SO₃H and H;
Y′ represents 1 to 4 groups independently selected from the group consisting of H, CH₂NH₂, SO₃H, CH₂COOH, NCS and F, and wherein the Y′ groups are placed in any of the positions of the aromatic ring;
Q′ is independently selected from the group of H, SO₃H, NH₂, COOH, ammonium, ester groups, benzyl and a group G;
I is an integer from 1 to 3;
and m is an integer from 1 to 5.

In a preferred embodiment,

X′ is selected from the group of —C(CH₃)₂ and C(CH₃)(CH₂)₄M, wherein M is a group
G, preferably succinimidyl ester, or M is SO₃H;
Y′ represents SO₃H, H or 1 to 4 F atoms;
Q′ is selected from a group G, and is most preferably succinimidyl ester, and SO₃H;
I is preferably 2 and m is preferably 3, 4 or 5.

Cyanine chromophores particularly suitable for use in the present invention are disclosed in U.S. Pat. No. 5,268,486 (Waggoner et al) and include, but are not limited to, the Cy Chromophores™: Cy 3, Cy 3B, Cy 3.5, Cy 5, Cy 5.5, Cy 7 and Cy 7.5.

The quencher agent is preferably a non-fluorescent chromophore. Suitable non-fluorescent quencher agents may be selected from 2,4-dinitrophenyl (DNP), 4-(4-dimethylaminophenyl)azobenzoic acid (DABCYL), 7-methoxycoumarin-4-yl)-acetyl (Mca) and non-fluorescent cyanine chromophores, e.g. as described in WO 99/64519 and WO02/29407.

Preferred quencher agents are cyanine chromophores comprising a substitutent which reduces the fluorescence emission of the quencher agent such that it is essentially non-fluorescent. More preferably, the quencher agent is a cyanine chromophore comprising at least one nitro group which reduces the fluorescence emission of the quencher agent. Particularly preferred non-fluorescent quencher agents for use in the invention are cyanine chromophores having the structure of formula (IV):

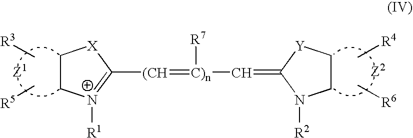

wherein groups R³, R⁴, R⁵ and R⁶ are attached to the rings containing X and Y or, optionally, are attached to atoms of the Z¹ and Z² ring structures and n is an integer from 1-3;
Z¹ and Z² each represent a bond or the atoms necessary to complete one or two fused aromatic rings each ring having five or six atoms, selected from carbon atoms and, optionally, no more than two oxygen, nitrogen and sulphur atoms;
X and Y are the same or different and are selected from bis-C₁-C₄ alkyl and C₄-C₅ spiro alkyl-substituted carbon, oxygen, sulphur, selenium, —CH═CH— and N—W wherein N is nitrogen and W is selected from hydrogen, a group —(CH₂)OR⁸ where o is an integer from 1 to 26 and R⁸ is selected from hydrogen, amino, aldehyde, acetal, ketal, halo, cyano, aryl, heteroaryl, hydroxyl, sulphonate, sulphate, carboxylate, substituted amino, quaternary ammonium, nitro, primary amide, substituted amide, and groups reactive with amino, hydroxyl, carbonyl, carboxyl, phosphoryl, and sulphydryl groups;
at least one of groups R¹, R², R³, R⁴, R⁵, R⁶ and R⁷ is a bonding group;
any remaining groups R³, R⁴, R⁵, R⁶ and R⁷ groups are independently selected from the group consisting of hydrogen, C₁-C₄ alkyl, OR⁹, COOR⁹, nitro, amino, acylamino, quaternary ammonium, phosphate, sulphonate and sulphate, where R⁹ is selected from H and C₁-C₄ alkyl;
any remaining R¹ and R² are selected from C₁-C₁₀ alkyl which may be unsubstituted or substituted with phenyl, the phenyl being optionally substituted by up to two substituents selected from carboxyl, sulphonate and nitro groups;
characterised in that at least one of the groups R₁, R₂, R₃, R₄, R₆ and R₇ comprises a substituent which reduces the fluorescence emission of said quencher agent such that it is essentially non-fluorescent.

Suitably, at least one of the groups R³, R⁴, R⁵, R⁶ and R⁷ of the non-fluorescent quencher is a nitro group which may be attached directly to the rings containing X and Y. Alternatively, a mono or di-nitro-substituted benzyl group may be attached to the rings containing X and Y, which optionally may be further substituted with one or more nitro groups attached directly to the aromatic rings.

The bonding group R¹, R², R³, R⁴, R⁵, R⁶ and R⁷ can be any group suitable for attaching the quencher to the enzyme clevable group, optionally via a linker. For example, the bonding group may be a reactive group or functional group G, as described for formula III, for reacting with the enzyme cleavable group.

Suitable pairs of fluorophores and quencher agents for use in the contrast agents of the invention are Cy3/Cy5Q, Cy3B/Cy5Q and Cy5/Cy7Q.

In a preferred embodiment the fluorophore is Cy5 and the non-fluorescent quencher agent is Cy7Q, both shown below.

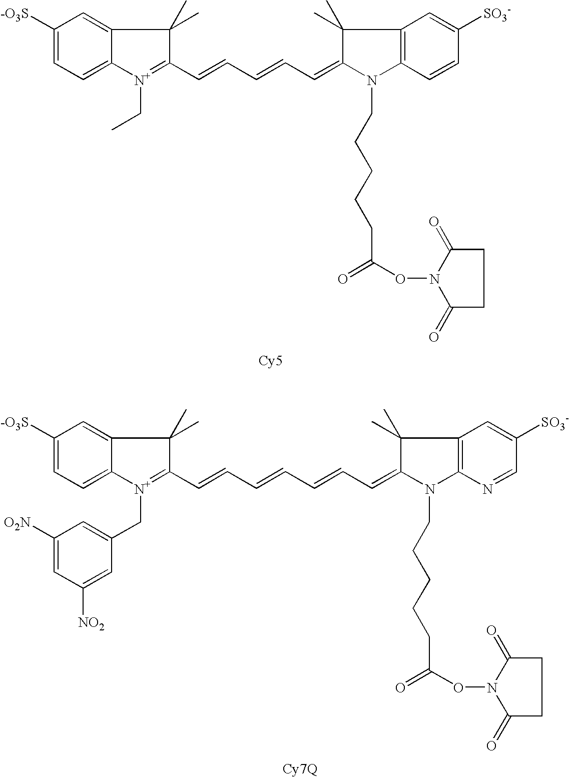

Energy transfer only takes place over short distances and therefor the fluorophore and quencher agent need to be in close proximity. In a preferred embodiment of the invention, the distance between the centres of Q and D in an energy transfer relationship is from 10 to 80 Angstroms, more preferably less than 60 Å.

The target binding ligand (V), also called a “vector” or “biological targeting moiety”, is a moiety which has affinity for biological receptors (“target”) associated with a given disease. The target binding ligand may be of synthetic or natural origin, but is preferably synthetic. The target binding ligand has the ability to direct the contrast agent to a region of a given disease. Preferably, the reaction of the target binding ligand with a receptor does not affect the remaining parts of the contrast agent, i.e. it does not affect the fluorogenic properties of the contrast agent. The target binding ligand has affinity for the receptor and preferably binds to this. On the one hand the target binding ligand should have a high affinity for the receptor, and on the other hand it should “stay” on the receptor as long as possible. Thus the target binding ligand should preferably exhibit slow dissociation kinetics. Preferred receptors are those receptors that are more than 50% more abundant in diseased tissue than in surrounding tissue. More preferred targets are those targets that are more than two times more abundant in diseased tissue than in surrounding tissue. The most preferred targets are those targets that are more than 5 times more abundant in diseased tissue than in surrounding tissue.

Relevant groups of receptors which the target binding ligand has affinity for are nucleic acids, proteins, including enzymes and inhibitors, lipids, other macromolecules as for example lipoproteins and glycoproteins. Preferred groups of receptors are proteins, lipoproteins and glycoproteins. The receptors may be localised in the vascular system, in the extracellular space, associated with cell membranes or localised intracellularly. Some enzymes may act as receptors when targeted by e.g. antibodies or peptides with affinity to the enzyme protein.

The target binding ligand can generally be any type of molecule that has affinity for a biological receptor. The molecules should be physiologically acceptable and should preferably have an acceptable degree of stability. The target binding ligands are e.g. selected from the following group of compounds: peptides, peptoids/peptidomimetics; oligonucleotides, such as Oligo-DNA or oligo-RNA fragments; oligosaccharides; lipid-related compounds; hormones; vitamins such as folate or biotin; neurotransmitters such as acetylcholine, serotonin or dopamine; synthetic small drug-like molecules; inhibitors; antibodies and antibody fragments; and derivatives and mimetics thereof. The target binding ligand may also act as an agonist or an inhibitor/antagonist.

Peptidic target binding ligands may be linear or cyclic, or combinations thereof. By the term “cyclic peptide” is meant a sequence wherein two amino acids are bonded together by a covalent bond which may be a peptide or disulphide bond or a synthetic non-peptide bond such as a thioether, phosphodiester, disoixane or urethane. The peptides may comprise 1, 2 or more such cyclic bridges and the number of amino acids between two amino acids which are bonded are e.g. 3-15. The peptides are preferably 3-100 mer peptides, and more preferably 3-30 mer peptides.

By the term “amino acid” is meant an L- or D-amino acid, amino acid analogue or amino acid mimetic which may be naturally occurring or of purely synthetic origin, and may be optically pure, i.e. a single enantiomer and hence chiral, or a mixture of enantiomers. Preferably the amino acids of the target binding ligand are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

Suitable peptides for use in the target binding ligand include the following, using standard symbols for the amino acids:
somatostatin, octreotide and analogues;
peptides which bind to the ST receptor, where ST refers to the heat-stable toxin produced by E. coli and other micro-organisms;
laminin fragments eg. YIGSR, PDSGR, IKVAV, LRE and KCQAGTFALRGDPQG;
N-formyl peptides for targeting sites of leukocyte accumulation;
Platelet factor 4 (PF4) and fragments thereof;
RGD-containing peptides;
Angiotensin II;
Endothelins;
Cytokines such as VEGF, EGF, hepatocyte growth factor, nerve growth factor, interferons, interleukins, platelet-derived growth factor, tumor necrosis factor, macrophage colony-stimulating factor and fragments thereof;
Chemokines such as MCP-1 and eotaxin;
Peptide fragments of α₂-antiplasmin, fibronectin or beta-casein, fibrinogen or thrombospondin.

Synthetic peptides of the target binding ligand may be obtained by conventional solid phase synthesis, as described by Merrifield employing an automated peptide synthesizer (J. Am. Chem. Soc., 85: 2149 (1964)).

Suitable oligonucleotides are polymers of ribonucleotides or deoxyribonucleotides comprising between 5 and 100 units, preferably between 10 and 30 units. The oligonucleotides may contain only the five common nitrogenous bases of natural nucleic acids, or they may contain unusual or synthetic bases. The bonds between the phosphorus atoms may be the natural oxygen ester bridges, or the oxygen may be replaced by another atom, such as carbon, nitrogen or sulphur in order to reduce the susceptibility of the oligonucleotides to hydrolysis by nucleases.

Suitable oligosaccharides are polymers of sugars, containing from three to twenty units, preferably from three to ten units. The constituent sugars are glucose, galactose, mannose, fructose, N-acetylglucosamine, N-acetylgalactosamine or sialic acids, but other sugars, including synthetically modified sugars, may be present. The sugar chains may be linear or branched.

Suitable lipid-related compounds are hydrophobic compounds with biological activity that may be the common building blocks of eukaryotic biological membranes, such as phospholipids, glycolipids or cholesterol. Preferably, they are related to or derived from these compounds. Examples of compounds that are derived from arachidonic acid are prostaglandins and thromboxanes. From phospholipids are derived lysophosphatidylcholine, diacylglycerol and platelet-activating factor; from cholesterol, steroids such as the cortisol, progesterone, estradiol and testosterone. Retinoids also belong in this general class of compounds.

Suitable enzyme inhibitors may be naturally occurring proteins such as cystatins, serpins or TIMPs (native or modified). They may be of microbial origin, such as leupeptin, semi-synthetic, or synthetic, such as lysine chloromethyl ketone.

Suitable monoclonal antibodies or fragments thereof for use in the present invention include: antibodies to the CD-20 antigen expressed on the surface of B-cells; anti-leucocyte or anti-granulocyte antibodies; anti-myosin antibodies or antibodies to carcinoembryonic antigen (CEA).

Suitable synthetic small drug-like small molecules for use in the present invention include: estradiol, estrogen, progestin, progesterone and other steroid hormones; ligands for the dopamine D-1 or D-2 receptor, or dopamine transporters such as tropanes; and ligands for the serotonin receptor.

The target binding ligand preferably has a molecular weight of less than 10000 Daltons, more preferably less than 4500 Daltons and most preferably less than 2500 Daltons.

In a preferred aspect the enzyme cleavable group of the contrast agents does not have an inhibitory or antagonistic effect, so the enzyme reacting with the enzyme cleavable group can perform its action repeatedly. Preferably, also the target binding ligand of the contrast agent is agonistic in the sense that it stimulates internalisation of the receptor in such a way that intracellular accumulation occurs and recycling of the receptor potentially occurs for further binding.

The contrast agent may comprise one or more linker moieties within the building blocks E-Q and V-D and/or between these. The function of the linker moieties is to connect the different parts of the contrast agent together, to obtain the right distance between Q and D to obtain the FRET relationship, and for preventing binding of the target binding ligand V in its non-activated form. The linker moieties L¹, L² and L³, as defined for formula I and II, are individually a linker group -(L)ₚ- where L is independently chosen from, —CR^{d}₂—, —CR^{d}═CR^{d}, —C≡C—, —NR^{d}CO⁻, —CONR^{d}—, —SO₂NR^{d}—, —NR^{d}SO₂—, —CR^{d}₂OCR^{d}₂—, —CR^{d}₂SCR^{d}₂—, —CR₂NR^{d}CR^{d}₂—, a C₄₋₈ cycloheteroalkylene group, a C₄₋₈ cycloalkylene group, a C₅₋₁₂ arylene group, a C₃₋₁₂ heteroarylene group or a polyalkyleneglycol, polylactic acid or polyglycolic acid moiety or an amino acid; wherein p is an integer of value 0 to 10 and each R^{d} group is independently H or C₁₋₁₀ alkyl, C₃₋₁₀alkylaryl, C₂₋₁₀ alkoxyalkyl, C₁₋₁₀ hydroxyalkyl, C₁₋₁₀ fluoroalkyl. When the linker group comprises one or several amino acids, preferred amino acids posses a functional side-chain such as an acid or amine group, e.g. aspartic or glutamic acid, homolysine or a diaminoalkylic acid such as lysine or diaminopropionic acid, more preferably aspartic acid or lysine. Alternatively, in the simplest form L is a functional bond or comprises a functional group X″ which permits facile conjugation of the building blocks, such groups including —NR^{d}—, CO₂, —N(C═S)—, —N(CO)—, —S, or —O—. Since most peptides and proteins have available carboxyl or amino sites for functionalisation, preferred X″ groups when the enzyme cleavable group E and/or the target binding ligand V is a peptide or protein are —NR^{d}, —CO₂, since these permit facile conjugation via amide bonds.

The contrast agents are designed for diseases where one receptor and one enzyme are co-jointly over-expressed in the same tissue or cells. In a preferred embodiment the contrast agent comprises a target binding ligand having affinity for a receptor selected from the group of receptor tyrosine kinase, such as VEGFR or EGFR, the family of Integrin receptors and Cancer Related Antigens, and an enzyme cleavable group having affinity for an enzyme selected from the group of matrix metalloproteinases, Cathepsins, Kallikreins, Proprotein convertases and Membrane bound serine proteases.

Examples of relevant receptors and enzymes upregulated in certain diseases are listed below. In one embodiment, the contrast agents of the invention react with one receptor and one enzyme from this list, upregulated in the same tissue or cells:

| Diseases | Upregulated receptors | Upregulated enzymes |
|---|---|---|
| Cancers | Vascular endothelial growth | Urokinase plasminogen activator, |
| generally | factor receptor (VEGFR) | Plasmin |
| | Integrin family: e.g. αvβ3, αvβ5 | MT-MMPs: MT1-MT6 & MMP- |
| | Epidermal growth factor receptor | 23 |
| | (EGFR), erbB-2, CD44 | Membrane-anchored serine |
| | | proteases: Prostatin, testisin, |
| | | TSP50, GPI-SP1-3, TESP 1-2, |
| | | DISP, testes serine proteases |
| | | 1,2, Tryptase-γ1,Matriptase-1 |
| | | (MT-SP1, epithin in the mouse), |
| | | corin, Matriptase-2 & 3, |
| | | enteropeptidase, hepsin, |
| | | TMPRSS 2, 3, 4, Spinesin, DESC |
| | | 1, 2, 3, HAT, MSPL |
| | | ADAMs: ADAM9, 10, 12, 15, 17 |
| | | Cathepsins: Cathepsin B |
| | | Kallikreins: human kallikrein-14 |
| | | (hK14) |
| | | matrix metalloproteinases |
| | | (MMPs): e.g. MMP-9 |
| | | Polyserase-1 |
| | | Heparanases |
| Breast | EGFR, erbB-2, TGFR, CD44, | Cathepsin D, proprotein |
| cancer | factor VIII-related antigen (FVIII- | convertases furin, PACE4, PC1, |
| | RA), human kallikrein-14 (hK14) | PC7, kallikrein-5 |
| | TIMP-1, PAI-1, FGF2R, IGFR, | |
| | HGFR, NGFRs | |
| Colorectal | Adhesion molecules and | Aminopeptidase N/CD13, matrix |
| cancer | adhesion-associated molecules: | metalloproteinases and their |
| | E-cadherin (CDH1 gene), CD44- | inhibitors (especially MMP-2, |
| | standard, CD44-6v, CD44-9v, 67- | MMP-7, MMP-9, MMP-14, |
| | kDa laminin receptor. | stromelysin-3), plasminogen- |
| | Antigens: | related molecules, u-PA, |
| | Human leukocyte antigen-B18 and | Cathepsins, typically |
| | human leukocyte antigen-DQ5, | Cathepsin B, proprotein |
| | tissue polypeptide antigen (TPA) | convertase PC5. |
| | or tissue polypeptide-specific | |
| | antigen (TPS), Small intestinal | |
| | mucin antigen (SIMA), CA15.3, | |
| | CA 19-9, CA 72-4, CEA, MUC-1, | |
| | tumour-associated antigen L6, | |
| | HLA-A, CA-195, CA-242, AFP, | |
| | CA125. | |
| | Enzymes: | |
| | aminopeptidase N/CD13, matrix | |
| | metalloproteinases and their | |
| | inhibitors (especially MMP-2, | |
| | MMP-7, MMP-9, stromelysin-3), | |
| | plasminogen-related molecules, u- | |
| | PA, Cathepsins, typically | |
| | Cathepsin B. | |
| | Signal molecules and their | |
| | receptors: | |
| | c-erbB2, VEGF, c-Myc, CCK(B)-R, | |
| | Bradeion (septin family gene), | |
| | benzodiazepine receptor, Her-2, | |
| | VEGF receptors, EGF receptors, | |
| | c-Met, neurotensin receptors. | |
| | Others: | |
| | VAMP2, Clusterin (apolipoprotein | |
| | j), ITF-2, osteopontin | |
| Prostate | Prostate specific antigen, | Hepsin, TMPRSS2, MMP-9, |
| cancer | VEGFR, CD44, syndecan-1, | kallikrein-11, kallikrein-2, MMP-2, |
| | prostate stem cell antigen, | MMP-9, PIM-1 gene product, |
| | cathepsin D, hepsin, MT1-MMP, | neutral endopeptidase |
| | uPAR, EGFR, Fas, FasL, | |
| | macrophage scavenger receptor-1 | |
| Lung | Adhesion molecules and | Caspase-9 and -3, MMPs (incl. |
| cancer | extracellular matrix proteins: | collagenase, MMP-9, |
| | CD44, CD44v3, CD44v6, ED-B | Stromelysin-3), urokinase |
| | fibronectin, galectin-3, galectin-4, | plasminogen activator, kallikrein |
| | LGALS3 (Galectin) gene product, | 11, cathepsin H, cathepsin L, |
| | P-selectin, liver-intestinal cadherin | tissue plasminogen activator, |
| | 17 and integrins, such as αᵥβ₃. and | pronapsin A, proprotein |
| | αᵥβ₅. | convertases furin, PACE4 and PC2 |
| | Antigens | |
| | CA 15.3, CA 72.4, cancer antigen | |
| | 125 (CA125), CA19-9, | |
| | carbohydrate antigen 549 (CA | |
| | 549), carcinoembryonic antigen | |
| | (CEA), CD105, CD24, CD34, | |
| | melanoma antigen E tumor- | |
| | associated antigen, MUC1 | |
| | (glycosylated mucin), squamous | |
| | cell carcinoma antigen (SCC), | |
| | tissue polypeptide antigen (TPA), | |
| | 5T4 oncofetal trophoblast | |
| | glycoprotein, FOS-related antigen | |
| | 1, H/Le^{y}/Le^{b}. | |
| | Oncogenes | |
| | c-erbB-2, EphA2 receptor tyrosine | |
| | kinase, HER2/EGFR | |
| | Signalling receptors | |
| | Cholecystokinin A receptor, | |
| | Cholecystokinin B receptor, | |
| | EGFR tyrosine kinase, EGFR, | |
| | Notch3, TIE-2 precursor, c-myc | |
| | protein, Gastrin-releasing peptide | |
| | receptor, neuromedin B receptor, | |
| | bombesin receptor,, neurotensin | |
| | receptor, uPAR, vasopressin | |
| | receptor, the angiopoietin | |
| | receptors, VEGFR, bradykinin | |
| | receptor. | |
| | Enzymes | |
| | carbonic anhydrase I and II, | |
| | carbonic anhydrase-9, caspase-9 | |
| | and -3, MMP (collagenase, MMP- | |
| | 9, Stromelysin-3), | |
| | myeloperoxidase, urokinase | |
| | plasminogen activator, kallikrein | |
| | 11, cathepsin H, cathepsin L, | |
| | tissue plasminogen activator, | |
| | pronapsin A,, carboxypeptidase | |
| | E, proprotein convertase | |
| Carcinoma | EGFR, erbB2, CD44, CD44H, | Cathepsin D, DESC-1 (squamous |
| of the | CD44v2, CD44V6, c-myc, c-Met, | cell carcinoma), proprotein |
| oesopha- | guanylyl cyclase, MUC1, | convertases furin, PACE4, |
| gus | MUC5AC, squamous cell | MMP-1, MMP-2, MMP-7, MMP- |
| | carcinoma antigen, | 9, MMP-12, MMP-14 |
| | β-catenin, cholecystokinin | |
| | receptors A and B, SCC, Tumor | |
| | M2-PK, c-erb2, integrins αᵥβ₃ and | |
| | αᵥβ₅, ligands of Helix pomatia | |
| | lectin, MUC4, Epidermal growth | |
| | factor receptor (EGFR), MMP-1, | |
| | MMP-2, MMP-7, MMP-9, MMP- | |
| | 12, MMP-14, Cathepsin D | |
| Athero- | Collagens I, III and IV, endothelin | MMP-3 & 9, elastase, |
| sclerotic | receptors, angiotensin II | collagenases, cathepsin B |
| plaque | receptors, CD36, CD40, C- | cathepsin K, urokinase, ADAMs, |
| | reactive protein, SR-A, SR-B1, | |
| | Toll-like receptor 4, VEGFR, | |
| | LOX-1, Factor VIII. | |

In an even more preferred embodiment the contrast agent of the invention has affinity to any of the following pairs of enzymes and receptors:
E.g. in tumour angiogenesis: An MMP, such as MMP-9, and VEGFR; MT-1 metalloproteinase and αᵥβ₃ integrin
E.g. in breast cancer: MMP-9 and EGFR; kallikrein-14 and CD44; cathepsin D and nerve growth factor receptors
E.g. in squamous cell carcinoma of the oesophagus: cathepsin D and EGFR; MMP-9 and MUC5AC
E.g. in adenocarcinoma and other carcinomas of the oesophagus: MMP-12 and cholecystokinin receptors; cathepsin D and guanylyl cyclase
E.g. in lung cancer: galectin-3 and MMP-9; urokinase plasminogen activator and CA125; cathepsin L and cholecystokinin receptors
E.g. in prostate cancer: hepsin and prostate stem cell antigen; hepsin and EGFR; cathepsin D and prostate-specific antigen
E.g. in colorectal cancer: aminopeptidase N/CD13 and CEA; MMP-9 and c-Myc; cathepsin B and clusterin; uPA and benzodiazepine receptor; MMP-14 and c-Met.
E.g. in atherosclerotic plaque: MMP-3 and endothelin receptors; MMP-9 and CD36; cathepsin B and C-reactive protein

The contrast agents of the invention can be synthesized using known methods of chemical synthesis. The contrast agents may be prepared by covalent binding of the fluorophore and quencher agents to the target binding ligand and the enzyme cleavable group, using direct chemical coupling methods that are well known to the skilled person. Group Q may be initially attached to E, and D may be attached to V before conjugation of these two building blocks. Alternatively, V and E are initially coupled, optionally via a linker, prior to coupling of the fluorophore and quencher agent to this building block.

Appropriate fluorophore and quencher agents, such as the cyanine dyes Cy5 NHS ester (PA15101) and Cy7Q NHS ester (PA77101) are commercially available from GE Healthcare, formerly Amersham Biosciences. Target binding ligands and enzyme cleavable groups are commercially available (e.g. Sigma-Aldrich), or they may be extracted from biological materials or can be synthesised.

When the target binding ligand and/or the enzyme cleavable group is a peptide the solid-phase methodology of Merrifield employing an automated peptide synthesizer (J. Am. Chem. Soc., 85: 2149 (1964)) is particularly useful. In addition, coupling of the fluorophore and quencher agent can also be carried out automatically yielding e.g. an amide bond between the different components. Typically, the desired sequences are assembled by solid-phase peptide synthesis. Standard procedures for the synthesis strategy employed for the examples of this invention are described in E. Atherton & R. C. Sheppard, “Solid phase peptide synthesis: a practical approach”, 1989, IRL Press, Oxford.

For example, a resin with an acid-labile linker group, to which the desired amino-protected C-terminal amino acid residue has been esterified, is used. The amino protecting group is then removed and the second amino acid in the sequence is coupled using a suitable condensation reagent. Amino acids with semi-permanent amino protecting groups and permanent protecting groups for the functional side chains are employed. Amino-deprotection and coupling cycles are then repeated in alternating steps until the sequence of interest is assembled.

Alternatively, the peptides can be synthesised through solution peptide synthesis methods known in the art, either in a step-wise manner from the carboxyl terminus and/or through the application of segment condensation or ligation methods, employing comprehensive or minimal protection strategies. Combined solution-solid phase segment condensation approaches can also be applied.

Generally, the reactive side-chain groups present in the amino acids (for example amino, hydroxyl, guanidino and carboxyl groups) will be protected during overall synthesis as indicated above. A wide choice of protecting groups for amino acids is known (see, e.g., Greene, T. W. & Wuts, P. G. M. (1991) Protective groups in organic synthesis, John Wiley & Sons, New York). Amino protecting groups which may be employed include 9-fluorenylmethoxycarbonyl (Fmoc) and t-butyloxycarbonyl (Boc). Side-chain protecting groups which may be employed include t-butyl (tBu), trityl (Trt), Boc, and 2,2,5,7,8-pentamethylchroman-6-sulfonyl (Pmc). It will be appreciated that a wide range of other such groups are known in the art.

Finally the permanent side-chain protecting groups are removed and the peptide is cleaved from the resin, usually simultaneously through treatment with a suitable acidic reagent, e.g. trifluoroacetic acid (TFA).

Peptides used in the invention containing multiple disulfide bridges are synthesised using different cysteine protecting groups so that no ambiguity exists as to the final folded form of the ligand. The synthesis disclosed in WO03/006491, describing how the peptides, including thioether and disulphide bridges are formed, may be used.

Peptides, proteins and oligonucleotides for use in the invention may be labelled with fluorophores and quencher agents at a terminal position, or alternatively at one or more internal positions. For reviews and examples of protein labelling using fluorescent dye labelling reagents, see “Non-Radioactive Labelling, a Practical Introduction”, Garman, A. J. Academic Press, 1997; “Bioconjugation—Protein Coupling Techniques for the Biomedical Sciences”, Aslam, M. and Dent, A., Macmillan Reference Ltd, (1998). Protocols are available to obtain site specific labelling in a synthesised peptide, for example, see Hermanson, G. T., Bioconjugate Techniques, Academic Press (1996). Conjugation of a fluorophore and a quencher agent to a peptide can be accomplished by known methods of chemical synthesis. The nucleophile substitution reaction where a leaving group on the peptide N-terminus is replaced by a nucleophilic group on the fluorophore and/or quencher agent can be used. Particularly useful is the reaction between a cyanine dye active ester and a primary amino group in the peptide yielding an amide bond between the peptide and the chromophore moiety. Other linkages between the chromophore and the peptide, such as thioether or sulphone amide linkages may be obtained automatically, or the reaction of the chromophore and the peptide may be carried out by ordinary manual chemical synthesis. An amide linkage is e.g. formed from reaction between an amine and carboxylic group, a sulphonamide linkage is e.g. formed from reaction between an amine and an activated sulphonic acid, and a thioether linkaged is e.g. formed from reaction between a thiol and a halide.

Peptidic target binding ligands, peptidic enzyme cleavable groups and peptide-based contrast agents may be purified using high performance liquid chromatography (HPLC) and characterised by mass spectrometry and analytical HPLC before testing in the in vitro screen.

The contrast agents of the invention are intended for use in optical imaging. Any method that forms an image for diagnosis of disease, follow up of disease development or for follow up of disease treatment based on interaction with light in the electromagnetic spectrum from ultraviolet to near-infrared radiation falls within the term optical imaging. Optical imaging further includes all methods from direct visualization without use of any device and use of devices such as various scopes, catheters and optical imaging equipment, for example computer based hardware for tomographic presentations. The contrast agents will be useful with optical imaging modalities and measurement techniques including, but not limited to: luminescence imaging; fluorescence endoscopy; transmittance imaging; time resolved transmittance imaging; confocal imaging; nonlinear microscopy;-acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching. Methods based on measurement of properties of light emitted by fluorophores are preferred.

Viewed from another aspect, the invention provides a method including generating an image of a human or animal body by diagnostic imaging involving administering a contrast agent as described to the body, and generating an image of at least a part of the body, to which the contrast agent has distributed. While the present invention is particularly suitable for methods involving parenteral administration of the contrast agent, e.g. into the vasculature or directly into an organ of muscle tissue, intravenous administration being especially preferred, it is also applicable where administration is not via a parenteral route, e.g. where administration is transdermal/topical, nasal, sub-lingual or is into an externally voiding body cavity. The present invention is deemed to extend to cover such administration.

Viewed from a further aspect the invention provides a method of generating optical images of at least part of a human or animal body, previously administered with a contrast agent as defined.

Viewed from a still further aspect the invention provides a method of monitoring the effect of treatment of a human or animal body with a drug to combat a condition, the method involving administering to the body a contrast agent as described and detecting signal from the activated contrast agent taken up by cell receptors, said administration and detection optionally but preferably being effected repeatedly, e.g. before, during and after treatment with said drug. Said detection comprises an optical imaging technique.

Use of the contrast agents in imaging is hence an aspect of the invention. A preferred aspect is contrast agents as described for use in imaging, diagnosing, for surgical guidance and for monitoring the effect of treatment. Relevant indications wherein the contrast agents are useful are different forms of cancer and metastasis, e.g. breast, skin, colorectal, pancreatic, prostate, lung, stomach, esophageal, bladder or ovarian cancer. Alternatively, the contrast agent may be used for detection of diseases where activated macrophages are present such as vulnerable plaque in atherosclerosis and in inflammations. In the context of this invention, diagnosing includes screening of selected populations, early detection, biopsy guidance, characterisation, staging and grading. Monitoring the effect of treatment includes therapy efficacy monitoring and long-term follow-up of relapse. Surgical guidance includes tumour margin identification and nerve localisation during resection and sentinel lymph node detection.

The present invention also provides a pharmaceutical composition comprising an effective amount, e.g. an amount effective for enhancing image contrast in in vivo imaging of a contrast agent of the invention, or a salt thereof, together with one or more pharmaceutically acceptable adjuvants, excipients or diluents for example stabilizers, antioxidants, osmolality adjusting agents, buffers, pH adjusting agents, etc. The most preferred formulation is a sterile solution for intravascular administration or for direct injection into area of interest. Where the agent is formulated in a ready-to-use form for parenteral administration, the carrier medium is preferably isotonic or somewhat hypertonic.

Viewed from a further aspect the invention provides the use of a contrast agent of the invention for the manufacture of a contrast enhancing agent for use in a method of diagnosis involving administration of the contrast enhancing agent to a human or animal body and generation of an optical image of at least part of said body.

### BRIEF DESCRIPTION OF THE FIGURES

FIG. 1 illustrates one contrast agent of the invention and its action in vivo when reacting with an enzyme and a receptor.

FIG. 2 provides the compound structure of Example 3.

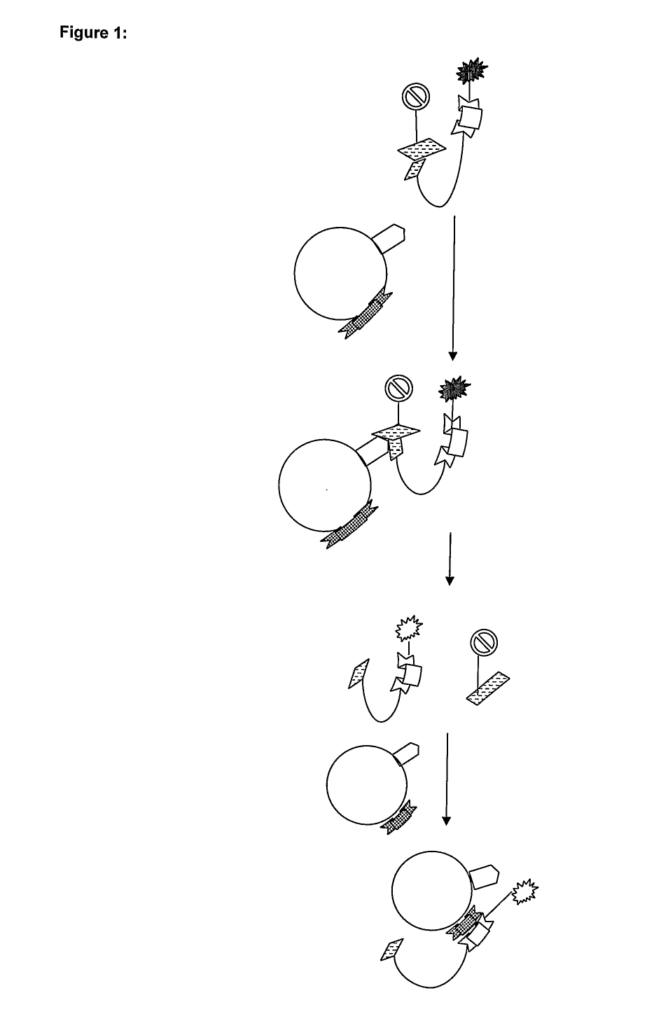

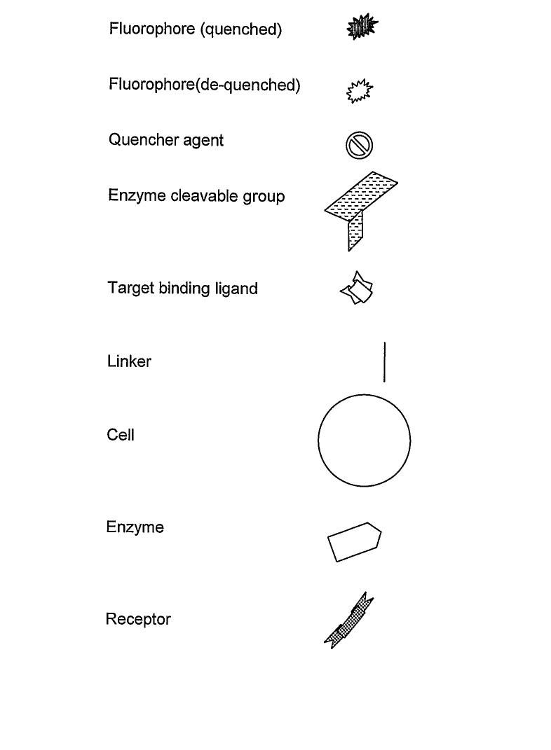

The present invention will now be further illustrated by way of the following non-limiting examples.

### EXAMPLES

### Example 1

### Synthesis of Ac-Thr-Met-Gly-Phe-Thr-Ala-Pro-Arg-Phe-Pro-His-Tyr-Lys(Cy5)-Ile-Pro-Gln-Gly-Leu-Leu-Gly-Lys(Cy7Q)-NH₂

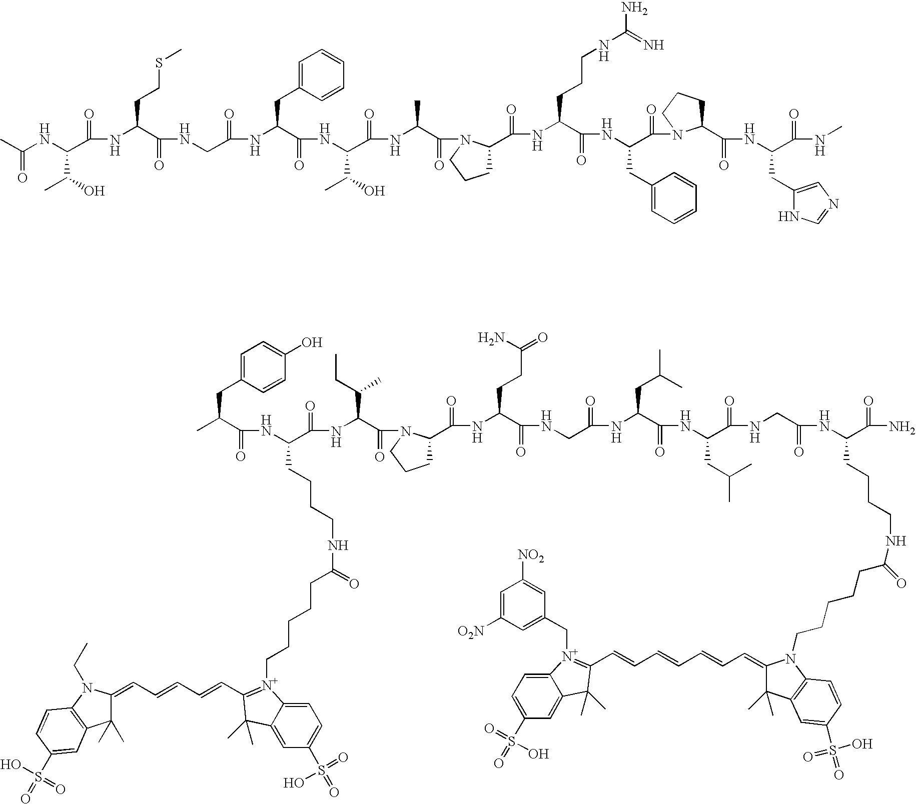

The peptide Thr-Met-Gly-Phe-Thr-Ala-Pro-Arg-Phe-Pro-His-Tyr is disclosed in WO02/02593, Seq. Id. no. 1, claimed to bind to the c-Met receptor. The peptide Ile-Pro-Gln-Gly-Leu-Leu-Gly is an MMP-14 substrate described by Ohkudo et al., Biochem. Biophys. Res. Comm. 1999, 266, 308-313. Cy5 is a fluorophore and Cy7Q is a quencher agent. Standard three-letter abbreviations for the amino acids are used.

Automated peptide synthesis was undertaken on an Applied Biosystems 433A peptide synthesizer using a piperidine-labile 9-fluorenylmethoxy-carbonyl/tert-butyl (Fmoc/tBu) coupling strategy. Rink Amide Novagel resin was used as solid phase support with the following coupling conditions applied:

To the resin (0.25 mmol, 0.59 mmol/g), was added excess amino acid (1 mmol) in N-methylpyrrolidone (NMP) followed by 1 mmol of coupling reagent O-Benzotriazol-1-yl-N,N, N′,N′-tetramethyluronium hexafluorophosphate/1-Hydroxybenzotriazole (HBTU/HOBt, 0.1M/0.45M) in dimethylformamide (DMF) and diisopropylethylamine (DIPEA) (2M, 1 ml). Deprotection of Fmoc residues was achieved using a 22% piperidine solution in NMP and monitored via a conductivity trace for completion. For preparative HPLC an AKTA Explorer instrument was used with a Vydac C18 21.5×250 mm protein and peptide column, eluent A was 0.1% TFA/Water and eluent B was acetonitrile. Analytical HPLC was undertaken on an AKTA Explorer instrument using a Vydac C18 4.6×250 mm protein and peptide column, eluent A was 0.1% TFA/Nater and eluent B was acetonitrile. MALDI-TOF mass spectra analysis was performed using a Kratos Kompact instrument and α-cyano-4-hydroxycinnamic acid as matrix. Electronic spectroscopy utilized a Unicam UV2 UV/Vis spectrometer.

All amino acids, with the exception of residue Lys²¹ which is 1-(4,4-dimethyl-2,6-dioxocyclohexylidene)-3-methylbutyl (ivDde) protected; are protected using standard side chain protecting groups compatible with the Fmoc strategy. The N-terminus was acetylated using an automated capping cycle and a solution of 0.5M acetic anhydride, 0.125M DIPEA and 0.015M HOBT in NMP.

Following solid phase automated synthesis, the resin bound peptide was isolated in quantitative yield (1.27 g). The simultaneous removal of side-chain protecting groups (except ivDde) and cleavage of the peptide from the resin was carried out on 330 mg of resin (65 μmol peptide) with 3.3 ml of trifluoroacetic acid (TFA) containing 2.5% triisopropylsilane (TIS), and 2.5% water for one hour. Precipitation of the peptide was induced by dropwise addition to diethyl ether (40 ml). The peptide was further washed in ether and collected via centrifugation to yield 130 mg (76%) of dried crude product. The crude product was purified by preparative RP-HPLC and lyophilised for the next step.

The peptide Ac-Thr-Met-Gly-Phe-Thr-Ala-Pro-Arg-Phe-Pro-His-Tyr-Lys-Ile-Pro-GIn-Gly-Leu-Leu-Gly-Lys(ivDde)-NH₂ (40 mg, 1.5×10⁵ mol) was dissolved in N-methylpyrrolidone (NMP) (10 mg/ml) together with DIPEA (160 ul) and Cy5 N-hydroxysuccinimidyl (NHS) ester (17 mg, 2.15×10⁻⁵ mol). The reaction mixture was stirred in an inert atmosphere in the absence of light for 18 hours before precipitation in ether (40 ml) and collection via centrifugation. The product was purified by preparative RP-HPLC and solvents removed in vacuo.

Ac-Thr-Met-Gly-Phe-Thr-Ala-Pro-Arg-Phe-Pro-His-Tyr-Lys(Cy5)-Ile-Pro-Gln-Gly-Leu-Leu-Gly-Lys(ivDde)-NH₂ was dissolved in dimethylformamide (DMF) containing 2% hydrazine monohydrate (5 ml). The reaction mixture was stirred under an inert atmosphere in the absence of light for 1 hour until all ivDde protecting groups were removed. The DMF solution was then added to diethyl ether to induce precipitation of the peptide. The material was washed by 3 cycles of diethyl ether addition and centrifugation before the product was dried for the final step.

Ac-Thr-Met-Gly-Phe-Thr-Ala-Pro-Arg-Phe-Pro-His-Tyr-Lys(Cy5)-Ile-Pro-Gln-Gly-Leu-Leu-Gly-Lys-NH₂ was dissolved in NMP (4 ml) together with DIPEA (160 ul) and Cy7Q NHS ester (22.5 mg, 2.15×10⁻⁵ mol.). The reaction was stirred in the absence of light for 18 hours until complete conjugation of Cy7Q was obtained. The reaction mixture was added to diethyl ether (40 ml) to induce precipitation of the peptide which was then collected via centrifugation. The final product Ac-Thr-Met-Gly-Phe-Thr-Ala-Pro-Arg-Phe-Pro-His-Tyr-Lys(Cy5)-Ile-Pro-Gln-Gly-Leu-Leu-Gly-Lys(Cy7Q)-NH₂ was purified by preparative RP-HPLC, lyophilised and analysed to yield 200 ug (0.35%) of product. Analytical HPLC (210, 650 and 700 nm), 89%. MALDI-TOF MS m/z 3859.7 (M+H⁺) ion observed. Electronic spectra (50% water/acetonitrile) 652 nm, 748 nm.

### Example 2

### Synthesis of Ac-Asp-Cha-Phe-D-Ser-D-Arg-Tyr-Leu-Trp-Ser-Lys(Cy3)-Gly-Pro-Leu-Pro-Leu-Arg-Ser-Trp-Lys(Cy5Q)-NH₂

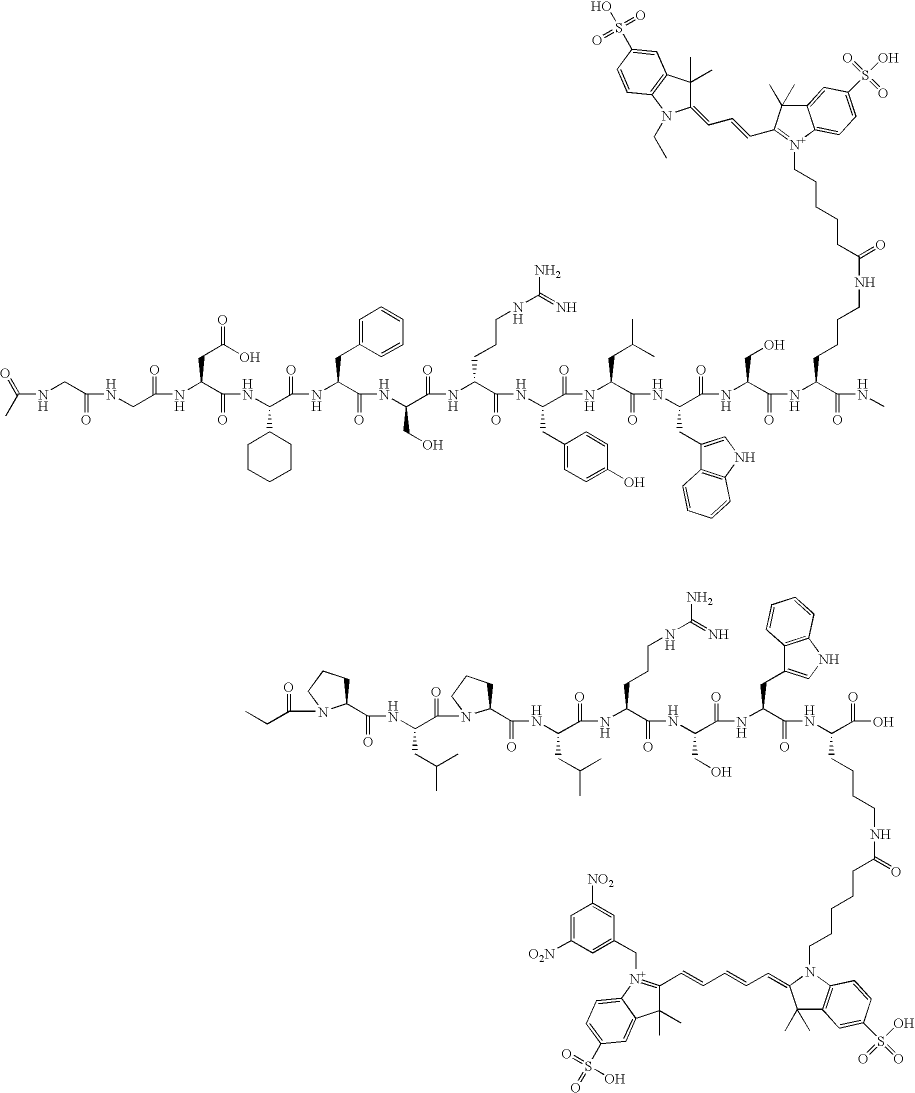

The peptide Asp-Cha-Phe-D-Ser-D-Arg-Tyr-Leu-Trp-Ser is disclosed in Ploug et al., Biochemistry 2001, 40, 12157-12168, claimed to bind to the uPA receptor. The peptide Gly-Pro-Leu-Pro-Leu-Arg-Ser-Trp is an MMP-2 substrate described by Ohkudo et al., Biochem. Biophys. Res. Comm. 1999, 266, 308-313. Cy3 is a fluorophore and Cy5Q is a quencher agent. Standard three-letter abbreviations for the amino acids are used. Cha is β-cyclohexyl-L-alanine.

The peptide is assembled on an Applied Biosystems 433A peptide synthesizer using piperidine-labile 9-fluorenylmethoxy-carbonyl/tert-butyl (Fmoc/tBu) strategy starting with 0.1 mmol Rink Amide Novagel resin. An excess of 1 mmol pre-activated amino acids, using O-Benzotriazol-1-yl-N,N,N′,N′-tetramethyluronium hexafluorophosphate (HBTU), is applied in the coupling steps. Residue Lys¹⁸ is 1-(4,4-dimethyl-2,6-dioxocyclohexylidene)-3-methylbutyl (ivDde) protected. All other amino acids are protected using standard protecting groups. The N-terminus is acetylated using a solution of 1 mmol acetic anhydride and 1 mmol N-methylmorpholine (NMM) in dichloromethane (DCM) for 60 minutes.

The simultaneous removal of side-chain protecting groups (except ivDde) and cleavage of the peptide from the resin is carried out in 10 mL trifluoroacetic acid (TFA) containing 2.5% triisopropylsilane (TIS) and 2.5% water for two hours. Trifluoroacetic acid is removed in vacuo, diethyl ether added to the residue and the precipitate washed with diethyl ether and air-dried affording crude product. The crude product is purified by preparative RP-HPLC and lyophilised.

The pure peptide Ac-Asp-Cha-Phe-D-Ser-D-Arg-Tyr-Leu-Trp-Ser-Lys-Gly-Pro-Leu-Pro-Leu-Arg-Ser-Trp-Lys(ivDde)-NH₂ is dissolved in N-methylpyrrolidone (NMP) (10 mg/ml) together with NMM (4 eq.) and Cy3 N-hydroxysuccinimidyl (NHS) ester (2 eq.). The reaction mixture is stirred in the dark until complete conjugation of Cy3 is obtained. NMP is evaporated in vacuo and the product purified by preparative RP-HPLC.

The pure peptide Ac-Asp-Cha-Phe-D-Ser-D-Arg-Tyr-Leu-Trp-Ser-Lys(Cy3)-Gly-Pro-Leu-Pro-Leu-Arg-Ser-Trp-Lys(ivDde)-NH₂ is dissolved in dimethylformamide (DMF) containing 2% hydrazine monohydrate. The reaction mixture is stirred until all ivDde protecting groups are removed. DMF is then evaporated in vacuo and the product purified by preparative RP-HPLC.

The pure peptide Ac-Asp-Cha-Phe-D-Ser-D-Arg-Tyr-Leu-Trp-Ser-Lys(Cy3)-Gly-Pro-Leu-Pro-Leu-Arg-Ser-Trp-Lys-NH₂ is dissolved in NMP (10 mg/ml) together with NMM (4 eq.) and Cy5Q NHS ester (2 eq.). The reaction mixture is stirred in the dark until complete conjugation of Cy5Q is obtained. NMP is evaporated in vacuo and the final product Ac-Asp-Cha-Phe-D-Ser-D-Arg-Tyr-Leu-Trp-Ser-Lys(Cy3)-Gly-Pro-Leu-Pro-Leu-Arg-Ser-Trp-Lys(Cy5Q)-NH₂ purified by preparative RP-HPLC.

### Example 3

### Synthesis of a Compound Comprising a Targeting Moiety to the VEGF Receptor, a Heparanase Substrate Sequence, a Fluorochrome and a Quencher

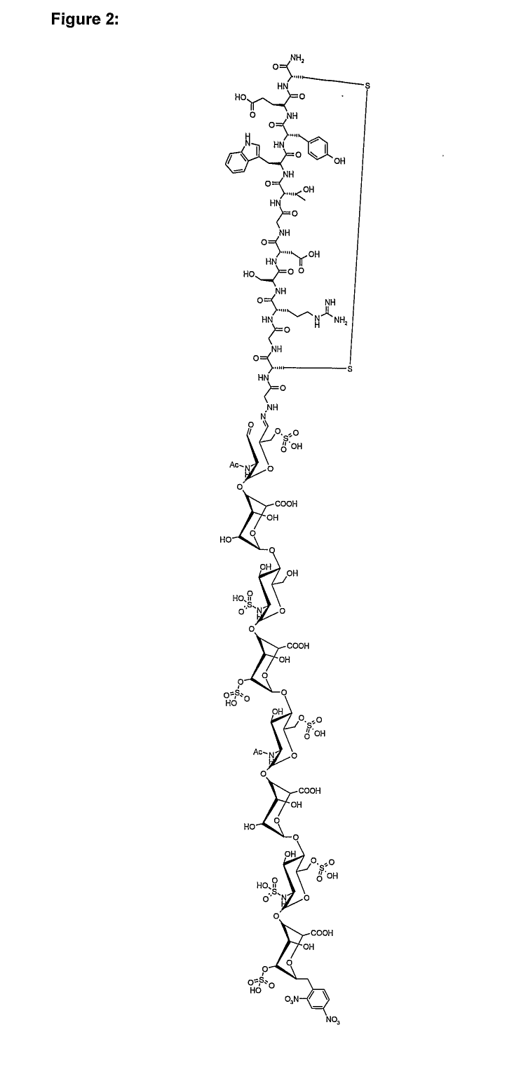

The compound structure is shown in FIG. 2.

The heparanase substrate sequence, an octasaccharide, is synthesised as described by Codée et al. (2004) and references therein, with modifications as outlined below. The synthesis proceeds by joining derivatised disaccharides comprising D-glucosamine and L-iduronic acid. The first disaccharide is attached to a solid support by way of the 6-carboxyl of L-iduronic acid.

In the present synthesis, the reducing end is protected as the 2,4-dinitrophenyl glucoside. After attachment of the final disaccharide, the N-acetylglucosamine-6-O-sulfate at the non-reducing end is oxidised with periodate under controlled conditions to give terminal aldehyde groups at the 3- and 4-carbons.

The peptide NH₂—NH-Cys-Gly-Arg-Ser-Asp-Gly-Thr-Trp-Tyr-Glu-Cys-NH₂ (disulfide bridge between Cys1-11) is a VEGF targeting peptide which has been disclosed in WO2004/058802. The peptide is assembled on an Applied Biosystems 433A peptide synthesizer using piperidine-labile 9-fluorenylmethoxy-carbonyl/tert-butyl (Fmoc/tBu) strategy starting with 0.1 mmol Rink Amide Novagel resin. An excess of 1 mmol pre-activated amino acids, using O-Benzotriazol-1-yl-N,N,N′,N′-tetramethyluronium hexafluorophosphate (HBTU), is applied in the coupling steps. All amino acids are protected using standard protecting groups. The N-terminus is hydrazinoacetylated using a solution of 1 mmol tri-Boc-hydrazinoacetic acid (Novabiochem), 1 mmol (7-Azabenzotriazole-1-yloxy)tripyrrolidinophosphonium hexafluorophosphate (PyAOP) and 2 mmol N-methylmorpholine (NMM) in dimethylformamide (DMF) for 60 minutes.

The simultaneous removal of side-chain protecting groups and cleavage of the peptide from the resin is carried out in 10 mL trifluoroacetic acid (TFA) containing 2.5% triisopropylsilane (TIS) and 2.5% water for two hours. Trifluoroacetic acid is removed in vacuo, diethyl ether added to the residue and the precipitate washed with diethyl ether and air-dried affording crude product. The crude product is purified by preparative RP-HPLC and lyophilised, then allowed to react with the oxidised octasaccharide to form a Schiff base. The final conjugate is dissolved in water and the solution adjusted to pH 8 to make the disulfide bridge.

The fluorescence from tryptophan is quenched by the dinitrophenyl group. The quenching is relieved on cleavage of the octasaccharide by heparanase. The fluorescence emission is read at 350 nm after excitation at 283 nm.

What is claimed is:

1.-17. (canceled)

18. A dual targeting optical imaging contrast agent suitable for imaging a disease state of the human or animal body, said contrast agent comprising:
a target binding ligand which has affinity for a biological receptor (V);
an enzyme cleavable group which is activated by an enzyme (E);
a fluorophore (D);
a quencher agent (Q);
conjugated with each other in one molecule;
wherein, as a result of said disease state, said biological receptor and said enzyme are co-jointly upregulated in the same tissue or cells.

19. A contrast agent as claimed in claim 18 comprising the building blocks
i) E-Q and
ii) V-D;
conjugated with each other,
wherein E, Q, V and D are as defined in claim 18.

20. A contrast agent as claimed in claim 18 of formula (I) or (II):
Q-L¹-E-L²-V-L³-D (I)
Q-L¹-E-L²-D-L³-V (II)
wherein Q, D, E and V are as hereinbefore defined, and L¹, L² and L³ are all linker moieties which are the same or different.

21. A contrast agent as claimed in claim 18, wherein E is cleaved by an enzyme prior to binding of V to a receptor.

22. A contrast agent as claimed in claim 18, wherein V has affinity for a receptor selected from the group of nucleic acids, proteins, lipids, lipoproteins and glycoproteins.

23. A contrast agent as claimed in claim 18, wherein V is selected from the group of peptides, peptoids/peptidomimetics, oligonucleotides, oligosaccharides, lipid-related compounds, synthetic small drug-like molecules, inhibitors, antibodies and antibody fragments, hormones, vitamins, neurotransmitters and derivatives and mimetics thereof.

24. A contrast agent as claimed in claim 18, wherein E comprises a substrate for an enzyme selected from the group of a protease, a peptidase, an esterase, a phosphatase, a phosphodiesterase, a dealkylase and a glycosidase or endoglycanase.

25. A contrast agent as claimed in claim 18, comprising a target binding ligand having affinity for a receptor selected from the group of Receptor tyrosine kinases, the family of Integrin receptors and Cancer Related Antigens, and an enzyme cleavable group having affinity for an enzyme selected from the group of matrix metalloproteinases, Cathepsins, Kallikreins, Proprotein convertases and Membrane bound serine proteases.

26. A contrast agent as claimed in claim 18, wherein Q is an acceptor chromophore and D is a donor chromophore in a FRET relationship, and wherein Q is essentially non-fluorescent.

27. A contrast agent as claimed in claim 18, wherein D is selected from the group of coumarin dyes, benzocoumarin dyes, xanthene dyes, phenoxazine dyes, rhodamines dyes, acridone dyes, merocyanine dyes, cyanine dyes and derivatives of the bis-pyrromethine boron difluoride chromophores.

28. A contrast agent as claimed in claim 18, wherein Q is selected from the group of 2,4-dinitrophenyl (DNP), 4-(4-dimethylaminophenyl)azobenzoic acid (DABCYL), 7-methoxycoumarin-4-yl)-acetyl (Mca) and non-fluorescent cyanine chromophores.

29. A pharmaceutical composition comprising an effective amount of a compound of claim 18, together with one or more pharmaceutically acceptable adjuvants, excipients or diluents.

30. A contrast agent or composition according to claim 18 for use as an optical imaging contrast agent.

31. Use of a contrast agent as claimed in claim 18 for the manufacture of an optical imaging contrast enhancing agent for use in a method of diagnosis involving administering said contrast agent to a human or animal body and generating an image of at least part of said body.

32. A method of generating images of a human or animal body by optical imaging involving administering a contrast agent as claimed in claim 18 to the body, and generating an image of at least a part of the body to which the contrast agent has distributed.

33. A method of monitoring the effect of treatment of a human or animal body with a drug to combat a condition, said method involving administering to said body a contrast agent or composition as claimed in claim 18 and detecting the uptake of said compound or composition by cell receptors, said administration and detection optionally but preferably being effected repeatedly, e.g. before, during and after treatment with said compound or composition.


SEQUENCE LISTING (13 sequences)

SEQ ID NO: 1 - protein, 5 aa, Artificial sequence
  Synthetic peptide
RGFFL

SEQ ID NO: 2 - protein, 5 aa, Artificial sequence
  Synthetic peptide
RGFFP

SEQ ID NO: 3 - protein, 11 aa, Artificial sequence
  Synthetic peptide
PKPQQFFGLKG

SEQ ID NO: 4 - protein, 10 aa, Artificial sequence
  Synthetic peptide
RPKPVEXWRK

SEQ ID NO: 5 - protein, 11 aa, Artificial sequence
  Synthetic peptide
XGPQGFQGNXG

SEQ ID NO: 6 - protein, 9 aa, Artificial Sequence
  Synthetic peptide
LRLSSYYSG

SEQ ID NO: 7 - protein, 5 aa, Artificial sequence
  Synthetic peptide
YIGSR

SEQ ID NO: 8 - protein, 5 aa, Artificial sequence
  Synthetic peptide
PDSGR

SEQ ID NO: 9 - protein, 5 aa, Artificial sequence
  Synthetic peptide
IKVAV

SEQ ID NO: 10 - protein, 15 aa, Artificial sequence
  Synthetic peptide
KCQAGTFALRGDPQG

SEQ ID NO: 11 - protein, 21 aa, Artificial sequence
  Synthetic peptide
TMGFTAPRFPHYKIPQGLLGK

SEQ ID NO: 12 - protein, 19 aa, Artificial sequence
  Syntesised peptide
DXFSRYLWSKGPLPLRSWK

SEQ ID NO: 13 - protein, 11 aa, Artificial sequence
  Synthesised peptide
CGRSDGTWYEC